(19) United States
(12) Patent Application Publication
(10) Pub. No.: US 2007/0286849 A1
(43) Pub. Date: Dec. 13, 2007

(54) TREATMENT OF AUTOIMMUNE DISORDERS

(75) Inventor: Vipin Kumar Chaturvedi, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(73) Assignee: Torrey Pines Institute for Molecular Studies, San Diego, CA (US)

(21) Appl. No.: 11/748,944

(22) Filed: May 15, 2007

Related U.S. Application Data
(60) Provisional application No. 60/801,533, filed on May 17, 2006.

(51) Int. Cl. A61K 35/26; A61P 37/00; A61P 37/06

(52) U.S. Cl. 424/93.71; 435/372.3

(57) ABSTRACT

Disclosed herein are methods for the treatment of autoimmune or immune related diseases or disorders. Also disclosed are methods for treating such autoimmune or immune related diseases or disorders with the administration of expanded populations of regulatory T cells. Also disclosed herein are methods of treating autoimmune or immune related diseases or disorders by administering an amount of expanded regulatory T cells to the body of a patient effective to reduce or prevent the symptoms of the autoimmune or immune related disease or disorder.

### CROSS-REFERENCE TO RELATED APPLICATIONS

This application claims priority under 35 U.S.C. § 119(e) to U.S. Provisional Application No. 60/801,533 filed May 17, 2006, which is hereby incorporated by reference in its entirety.

### BACKGROUND OF THE INVENTION

1. Field of the Invention

The present embodiments relate to methods for the treatment and prevention of autoimmune or immune related diseases or disorders. More specifically the present embodiments relate to methods of increasing the activation of certain immune-related cells in the body for use in the treatment and prevention of autoimmune or immune related diseases or disorders.

2. Description of the Related Art

Autoimmune diseases affect millions of people worldwide and can have devastating effects on lifespan and quality of life. Despite advances in medical science, many autoimmune diseases have evaded treatment because the mechanisms of disease are complex and poorly understood. Also, unlike most diseases where treatment involves working with the body's immune system to combat a foreign invader, in autoimmune diseases, the immune system itself is exacerbating the problem. This makes any treatment much more difficult because it must address and sometimes even combat the immune response directly to ameliorate the effects of the disease.

The immune system maintains a state of equilibrium while responding to foreign antigens as well as self-antigens. Control mechanisms for maintaining homeostasis following an immune response to a foreign antigen or for preventing or aborting harmful responses to self-antigens include CTLA-4-mediated T cell inhibition, activation-induced cell death (AICD), IL-2-mediated regulation, and regulatory T cells (T_{reg}) (¹Abbas, A. K., Lohr, J., Knoechel, B. & Nagabhushanam, V. T cell tolerance and autoimmunity. Autoimmun Rev 3, 471-5 (2004)). While much attention has recently been focused on CD4⁺CD25⁺ and NK T cells in suppressing the priming or expansion of T cell immunity (Sakaguchi, S. & Sakaguchi, N. Regulatory T cells in immunologic self-tolerance and autoimmune disease. Int Rev Immunol 24, 211-26 (2005)) (Kronenberg, M. Toward an understanding of NKT cell biology: progress and paradoxes. Annu Rev Immunol 23, 877-900 (2005)), less is known about the role of CD8⁺ T cells (CD8 T_{reg}) in feedback regulation of immunity (Gershon, R. K. & Kondo, K. Cell interactions in the induction of tolerance: the role of thymic lymphocytes. Immunology 18, 723-37 (1970)) (Jiang, H., Zhang, S. I. & Pernis, B. Role of CD8⁺ T cells in murine experimental allergic encephalomyelitis. Science 256, 1213-5 (1992)). Experiments using depleting antibodies or animals with a specific gene knockout have indicated an important regulatory role for CD8⁺ lymphocytes in autoimmune diseases (Koh, D. R. et al. Less mortality but more relapses in experimental allergic encephalomyelitis in CD8−/− mice. Science 256, 1210-3 (1992)), transplant tolerance (Seydel, K. et al. Anti-CD8 abrogates effect of anti-CD4-mediated islet allograft survival in rat model. Diabetes 40, 1430-4 (11991)), neonatal tolerance (Field, A. C., Caccavelli, L., Bloch, M. F. & Bellon, B. Regulatory CD8⁺ T cells control neonatal tolerance to a Th2-mediated autoimmunity. J Immunol 170, 2508-15 (2003)), and homeostasis of normal cellular and humoral immune responses (Nanda, N. K. & Sercarz, E. A truncated T cell receptor repertoire reveals underlying immunogenicity of an antigenic determinant. J Exp Med 184, 1037-43 (1996)). Thus the phenotype, antigen-specificity, and mechanisms utilized by CD8 T_{reg} are important not only for elucidating the biology of immune homeostasis but also for the development of strategies to manipulate immune responses.

In multiple sclerosis, for example, the immune system pathologically recognizes some self-antigens from myelin membranes as foreign and initiates an immune response against them. This results in demyelination, the destructive removal of myelin which is an insulating and protective fatty protein that sheaths nerve cells (neurons). The demyelination in multiple sclerosis is mediated by a T-cell guided immune response that is either initiated from antigen-presenting events in the CNS or induced following the peripheral activation by a systemic molecular mimicry response.

Experimental autoimmune encephalomyelitis (EAE) is a prototypic T-cell mediated autoimmune disease, characterized by inflammation and demyelination in the central nervous system accompanied by paralysis following immunization with myelin antigens, for example, myelin basic protein (MBP), myelin oligodendrocyte glycoprotein (MOG) or proteolipid protein (PLP). EAE shares many pathological and immune dysfunctions with human MS and is a widely accepted model for studying human MS.

Concanavalin A (Con A)-induced hepatitis in the mouse is a well-characterized model of T cell-mediated liver diseases. This model has been extensively used as an excellent model mimicking human T cell-mediated liver diseases, such as autoimmune hepatitis ((Tiegs et al., 1992, JCI, Mizuhara H., JEM, 1994, Toyabe S, J I, 1997). A single injection of Con A is sufficient for the T cell-mediated liver injury in mice (Tiegs et al., 1992, JCI, Mizuhara H., JEM, 1994, Toyabe S, J I, 1997). Serum enzymes and histological evidence of Con A induced hepatitis is observed following 8-24 hours, as shown by elevated serum levels of ALT and AST and the occurrence of histological evidence of hepatic lesions characterized by a massive granulocytes accumulation, CD4⁺ T cell infiltration and an influx of a relatively small number of CD8⁺ T cells and hepatocyte necrosis/apoptosis (Tiegs et al., 1992, JCI, Mizuhara H., JEM, 1994, Schumann J., 2000, Am. J. Pathol., Chen et al., 2001). Recently, several investigators have implicated hepatic NKT cells in the development of Con A-induced hepatitis. Both Jα18 and CD1d-deficient mice that lack NKT cells are resistant to Con A-induced hepatic injury (Kaneko et al., 2000; Takeda et al., 2000), indicating that classical CD1d-restricted NKT cells that express the iNKT cell receptor are critically involved in the process of Con A induced hepatic injury.

Another example of an autoimmune related disease or disorder is transplant rejection. During transplant rejection, a large number of T cells are activated, which pathogenically attack the transplanted organ. Immunosuppressants currently used to ameliorate the disease cause many damaging side effects for patients.

### SUMMARY OF THE INVENTION

Some embodiments relate to a method of treating, preventing, or delaying the onset of an autoimmune disease in a patient comprising administering isolated CD8αα⁺, TCRαβ⁺ cells to the patient.

Some embodiments related to obtaining a cell sample from a mammal; isolating CD8αα⁺, TCRαβ⁺ T cells from the cell sample; and expanding the isolated T cells.

In some embodiments, the isolated T cells are CD8αα⁺, TCRαβ⁺, CD200⁺.

In some other embodiments, the isolated T cells are CD8αα⁺, TCRαβ⁺, CD122⁺.

In still other embodiments, the isolated T cells are CD8αα⁺, TCRαβ⁺, CD200⁺, CD122⁺.

In some embodiments, the isolated T cells are a mixture of two or more of CD8αα⁺, TCRαβ⁺ T cells, CD8αα⁺, TCRαβ⁺, CD200⁺ T cells, CD8αα⁺, TCRαβ⁺, CD122⁺ T cells and CD8αα⁺, TCRαβ⁺, CD200⁺ CD122⁺ T cells.

In some embodiments, the cell sample comprises a blood sample.

In some embodiments, the cell sample comprises a tissue sample.

In some embodiments, the cell sample comprises lymph tissue.

In some embodiments, the mammal is the patient.

In some embodiments, the mammal is not the patient.

In some embodiments, the autoimmune disease is selected from the group consisting of multiple sclerosis, Crohn's disease, systemic lupus erythematosus, Alzheimer's disease, rheumatoid arthritis, psoriatic arthritis, enterogenic spondyloarthropathies, insulin dependent diabetes mellitus, autoimmune hepatitis, transplant rejection and celiac disease.

In some embodiments, the isolated T cells are isolated using antibodies.

In some embodiments, the antibodies are at least one of anti-TCR, anti-CD8αα, anti-CD200 and anti-CD122.

In some embodiments, the isolated T cells are expanded with growth factors.

In some embodiments, the isolated T cells are expanded with agents comprising anti-CD3 coated plates and one or more of IL-2, IL-7 and IL-15.

In some embodiments, the autoimmune disease is multiple sclerosis.

In some other embodiments, the autoimmune disease is transplant rejection.

In some embodiments, the T cells are administered to the patient by one or more of the routes consisting of intravenous, intraperitoneal, intramuscular, subcutaneous, nasal and oral.

In some embodiments, the T cells are administered to the patient by an intramuscular route.

In some embodiments, the patient is human.

In some embodiments, the T cells administered to the patient comprise about 20 million cells.

Some embodiments refer to an isolated T cell population, comprising an isolated population of T_{reg} cells characterized as CD8αα⁺, TCRαβ⁺.

In some embodiments, the T cell population is CD200⁺.

In some embodiments, the T cell population is CD122⁺.

In some embodiments, the T cell population is CD200⁺, CD122⁺.

In some embodiments the isolated T cell population is in combination with an aqueous vehicle and an additional pharmaceutically acceptable excipient.

In some embodiments the isolated T cell population is in combination with an aqueous vehicle and an additional pharmaceutically acceptable excipient.

In some embodiments the isolated T cell population is in combination with an aqueous vehicle and an additional pharmaceutically acceptable excipient.

In some embodiments the isolated T cell population is in combination with an aqueous vehicle and an additional pharmaceutically acceptable excipient.

### BRIEF DESCRIPTION OF THE DRAWINGS

FIG. 1 shows the proliferative and cytokine response of representative CD8 T cell clones.

FIG. 2 shows a panel of mAbs used to stain 2D11 to determine whether CD8 T_{reg} express characteristic cell-surface markers.

FIG. 3 shows the cytokine secretion profile of 2D11 as well as an OT-1 ovalbumin-specific CD8⁺ T cell clone (control).

FIG. 4 shows the proliferative response of 2D11 to p42-50 at titrated doses in the presence of APCs.

FIG. 5 shows mean disease scores of mice receiving different amounts of expanded T_{reg} cell populations in terms of the number of days after inducement of EAE with an injection of MBPAc1-9 (myelin basic protein).

FIG. 6 shows that T_{reg} clone 2D11 as well as a polyclonal CD 8 T_{reg} line expresses predominantly TCR Vβ6⁺ by flow cytometry, DNA sequencing and blocking of immune response in vitro using anti-Vβ6 antibody.

FIG. 7 shows that treatment of mice with agonistic anti-Vβ6 mAb in vivo results in activation of CD8αα⁺ Vβ6⁺ T cells and prevention of EAE.

### DETAILED DESCRIPTION OF THE INVENTION

The present embodiments are related to treatments for a wide variety of autoimmune or immune related diseases or disorders including, for example, multiple sclerosis, Crohn's disease, systemic lupus erythematosus, Alzheimer's disease, rheumatoid arthritis, psoriatic arthritis, enterogenic spondyloarthropathies, insulin dependent diabetes mellitus, autoimmune hepatitis, transplant rejection and celiac disease.

Some embodiments relate to newly isolated populations of T cells that are involved in the natural regulation cycle of the immune system. Regulatory T cells (T_{reg}) provide a balance to the immune response by killing other T cells that have expanded in response to a perceived antigen. Whether a foreign antigen or a self molecule has triggered an immune response, a population of T cells capable of attacking the antigen is generated and expanded. T_{reg} cells capable of killing the attacking T cells are then triggered to expand. This results in reduction in the population of the attacking T cells. In this way, an immune response can be efficient and directed, only lasting as long as necessary and doing as little collateral damage to any non-targeted tissues as possible.

In some embodiments, the newly isolated CD8αα⁺, TCRαβ⁺ T_{reg} cells are manipulated to treat the indications of autoimmune and immune related diseases and disorders. By isolating CD8αα⁺, TCRαβ⁺ T_{reg} cells from a patient, expanding the cells and then introducing the cells into body of the same or a different patient, the pathological self-reactive immune responses at the root of autoimmune and immune related diseases or disorders can be treated and reduced or eliminated.

CD8αα⁺, TCRαβ⁺ T_{reg} cells control the population of activated Vβ8.2⁺ CD4 T cells in vivo. These activated Vβ8.2⁺ CD4 T cells are pathogenically involved in EAE and other autoimmune diseases, attacking self antigens in the body and causing a variety of the often devastating symptoms of various diseases. These novel CD8αα⁺, TCRαβ⁺T_{reg} cells recognize a peptide from a conserved region of the T cell receptor (TCR) in the context of the class Ib MHC molecule, Qa-1a. This makes the T_{reg} cells specific for binding the pathogenic Vβ8.2⁺ CD4 T cells. Upon contact with the activated Vβ8.2⁺ CD4 T cells, the CD8αα⁺, TCRαβ⁺ T_{reg} cells secrete at least IFN-γ and kill only activated, Vβ8.2⁺ CD4⁺ T cells but not inactive Vβ8.2⁻ CD4⁺ T cells. This newly discovered specificity allows for therapeutic strategies against T-cell mediated diseases contemplated in some embodiments based on an important negative feedback regulatory loop that follows the activation of T cells in the body.

In some embodiments, the regulatory T cell population can be CD8αα⁺, TCRαβ⁺, CD200⁺ or CD8αα⁺, TCRαβ⁺, IL-2Rβ⁺ (CD122⁺) or CD8αα⁺, TCRαβ⁺, CD200⁺, CD122⁺. Each of CD8αα⁺, TCRαβ⁺, CD200⁺ T_{reg} cells, CD8αα⁺, TCRαβ⁺, CD122⁺ T_{reg} cells and CD8αα⁺, TCRαβ⁺, CD200⁺, CD122⁺ T_{reg} cells also control the population of activated Vβ8.2⁺ CD4 T cells in vivo and can be utilized in the same way as the CD8αα⁺, TCRαβ⁺ T_{reg} cells described above.

Some embodiments relate to methods for treating autoimmune or immune related diseases or disorders by first identifying a patient with an autoimmune or immune related disease or disorder and collecting a cell sample from the patient. The cell sample can be, for example, a blood sample, a tissue sample or lymph tissue. In some embodiments, the cell sample is a blood sample and once collected from the patient, peripheral blood leukocytes (PBLs) can then be isolated from the blood sample and stained, for example, with antibodies such as anti-TCR, anti-CD8αα, anti-CD200, anti-CD122, or any other antibodies specific to cell markers identified on T_{reg} cells or any combination of these antibodies. Then an isolation method, such as, for example, flow symmetry, bead chromatography or any other isolation method can be used to obtain T_{reg} cells. In some embodiments, the T_{reg} cells are CD8αα⁺, TCRαβ⁺ T cells, in other embodiments, the T_{reg}, cells are CD8αα⁺, TCRαβ⁺, CD200⁺ T cells, in still other embodiments, the T_{reg} cells are TCRαβ⁺, CD200⁺, CD122⁺ T cells and in other embodiments the T_{reg} cells are CD8αα⁺, TCRαβ⁺, CD200⁺, CD122⁺ T cells. Additionally, in some embodiments the isolated T_{reg} T cells can be expanded ex vivo or in vitro using any cell growth enhancing environment. The cell growth environment can be for example, anti-CD3 coated plates in the presence of growth factors such as IL-2, IL-7, IL-15, etc. In some embodiments, once the desired population of T cells has been grown up, they can then be transferred into the patient through any pharmaceutically acceptable route.

In some embodiments, the regulatory T cells can be administered to the same patient from which they were obtained. In other embodiments, the regulatory T cells can be administered to a patient other than the patient from which the they were obtained. In still other embodiments, the regulatory T cells can be obtained from a mammal that is not a patient. In other embodiments, the administered regulatory T cells can comprise a mixture of cells obtained from at least two of the patient to whom the regulatory T cells are administered, a patient other than the patient to whom the regulatory T cells are administered and a non-patient mammal.

In a preferred embodiment, anti-CD3 coated plates with growth factors such as IL-2, IL-7 and IL-15 are used to expand the T cell population. In other embodiments, T_{reg} can be expanded in vitro using recombinant TCR proteins or peptides, for example p42-50 derived from the TCR Vβ8.2 chain.

Some other embodiments relate to methods of treating autoimmune or immune related diseases or disorders by activating and expanding certain T cell populations within the body of a patient. For example, anti-TCR agents can be introduced into the body to expand or activate Vb6+ T_{reg} cells in vivo in a patient with an autoimmune related disease or disorder which results in the amelioration of the effects of the disease or disorder. In another embodiment, agents against the TCR, and against other cell surface markers on the T_{reg} population, such as, CD200, CD122, etc. can be used to activate T_{reg} cells in vivo, thereby reducing the population of pathogenic T cells and ameliorating the autoimmune related disease or disorder.

In another embodiment, the TCR Vβ or Vα chain gene utilized by disease-specific pathogenic T cells can be determined. Then the proteins corresponding to those TCR Vβ or Vα chain genes can be introduced into the body to activate the appropriate T_{reg} cell population.

Some embodiments are related to a method of treating the various indications of autoimmune or immune related diseases or disorders. In particular, one aspect of the present embodiment is related to a method of treating a patient suffering from symptoms of an autoimmune or immune related disease or disorder, such as, for example, multiple sclerosis, Crohn's disease, systemic lupus erythematosus, Alzheimer's disease, rheumatoid arthritis, psoriatic arthritis, enterogenic spondyloarthropathies, insulin dependent diabetes mellitus, autoimmune hepatitis, transplant rejection and celiac disease.

As used herein, the term “patient” refers to the recipient of a therapeutic treatment and includes all organisms within the kingdom animalia. In preferred embodiments, the animal is within the family of mammals, such as humans, bovine, ovine, porcine, feline, buffalo, canine, goat, equine, donkey, deer and primates. The most preferred animal is human.

As used herein, the terms “treat” “treating” and “treatment” include “prevent” “preventing” and “prevention” respectively. As used herein, the term “autoimmune disease” includes “immune-related disease,” “autoimmune disorder” “immunologic disorder” and “immune-related disorder.” As used herein, the term “isolated” refers to materials, such as cells or antibodies, which are removed from at least some of the components that normally accompany or interact with the materials in a naturally occurring environment such that they have been altered “by the hand of man” from their natural state to a level of isolation or purity that does not naturally occur. As used herein, the term “purified” refers to samples in which particular populations of T_{reg} cells are at least 10% or 20%, preferably 30% or 40% or more preferably 50% free from other components with which they are naturally associated. As used herein, the term “enriched” refers to samples in which the proportion of T_{reg} cells to other T cells is at least double, preferably 3 times, 5 times, 7 times 10 times, 15 times or 20 times that which occurs in a natural environment.

In some other embodiments, the expanded T_{reg} cell population can be administered alone or in combination with another therapeutic compound. Any therapeutic compound used in treatment of the target autoimmune disease can be used. In one embodiment, no adjuvant is used.

Many different modes and methods of administration of the therapeutic T_{reg} cell population are contemplated. In some embodiments, delivery routes include, for example, intravenous, intraperitoneal, inhalation, intramuscular, subcutaneous, nasal and oral administration or any other delivery route available in the art. Depending on the particular administration route, the dosage form may be, for example, solid, semisolid, liquid, vapor or aerosol preparation. The dosage form may include, for example, those additives, lubricants, stabilizers, buffers, coatings, and excipients available in the art of pharmaceutical formulations

Many pharmaceutical formulations are contemplated. In some embodiments, the pharmaceutical formulations can be prepared by conventional methods using the following pharmaceutically acceptable vehicles or the like: excipients such as solvents (e.g., water, physiological saline), bulking agents and filling agents (e.g., lactose, starch, crystalline cellulose, mannitol, maltose, calcium hydrogenphosphate, soft silicic acid anhydride and calcium carbonate); auxiliaries such as solubilizing agents (e.g., ethanol and polysolvates), binding agents (e.g., starch, polyvinyl pyrrolidine, hydroxypropyl cellulose, ethylcellulose, carboxymethyl cellulose and gum arabic), disintegrating agents (e.g., starch and carboxymethyl cellulose calcium), lubricating agents (e.g., magnesium stearate, talc and hydrogenated oil), stabilizing agents (e.g., lactose, mannitol, maltose, polysolvates, macrogol, and polyoxyethylene hydrogenated castor oil), isotonic agents, wetting agents, lubricating agents, dispersing agents, buffering agents and solubilizing agents; and additives such as antioxidants, preservatives, flavoring and aromatizing agents, analgesic agents, stabilizing agents, coloring agents and sweetening agents.

If necessary, glycerol, dimethyacetamide, 70% sodium lactate, surfactants and alkaline substances (e.g., ethylenediamine, ethanol amine, sodium carbonate, arginine, meglumine and trisaminomethane) can also be added to various pharmaceutical formulations.

In the context of some embodiments, the dosage form can be that for oral administration. Oral dosage compositions for small intestinal delivery include, for example, solid capsules as well as liquid compositions which contain aqueous buffering agents that prevent the expanded T_{reg} cell population or other ingredients from being significantly inactivated by gastric fluids in the stomach, thereby allowing the expanded T_{reg} cell population to reach the small intestines. Examples of such aqueous buffering agents which can be employed in the present invention include, for example, bicarbonate buffer at a pH of from about 5.5 to about 8.7. Tablets can also be made gastroresistent by the addition of, e.g., cellulose acetate phthalate or cellulose acetate terephthalate.

In some embodiments, the specific amount of the expanded T_{reg} cell population administered to a patient will vary depending upon the disease or condition being treated, as well as the age, weight and sex of the patient being treated. Generally, to achieve an effective final concentration in, e.g., the intestines or blood, the amount of the expanded T_{reg} cell population in a single dosage composition of the present invention will generally be from about 10,000 to about 1 trillion cells, preferably from about 100,000 to about 100 million cells, more preferably from about 1 million to about 50 million cells, even more preferably from about 10 million to about 30 million cells, even more preferably from about 15 million to about 25 million cells, and even more preferably about 20 million cells. Likewise, the amount of a secondary therapeutic compound in a single oral dosage composition of the present embodiments will generally be in the range of about 0.01 milligrams to about 1000 milligrams, more preferably about 0.1 milligrams to about 100 milligrams. Obviously, the exact dosage will vary with the disease or disorder being treated, the preferred ranges being readily determinable.

In another embodiment, the expanded T_{reg} cell population can be combined with a pharmaceutically acceptable vehicle. Suitable pharmaceutically acceptable vehicles include, for example, phosphate buffered saline. In one embodiment, from about 1,000 to about 3,000,000 cells/kg body weight of T_{reg} cells are administered to the patient. Preferably, from about 100,000 to about 1 million cells/kg body weight of T_{reg} cells are administered. This dosage can be repeated as needed on an hourly, daily, weekly, monthly or sporadic basis. Exemplary dosages and dose schedules are discussed infra.

In the present embodiments, the expanded T_{reg} cell population can be administered to a patient suffering from an autoimmune or immune related disease or disorder to improve the patient's condition. Accordingly, patients suffering from one or more of the various indications of an autoimmune or immune related disease or disorder such as multiple sclerosis, systemic lupus erythematosus, Alzheimer's disease, rheumatoid arthritis, insulin dependent diabetes mellitus, autoimmune hepatitis, transplant rejection and celiac disease can be treated with an expanded T_{reg} cell population according to the present embodiments.

In accordance with the embodiments, the expanded T_{reg} cell population can be administered to alleviate a patient's symptoms, or can be administered to counteract a mechanism of the disorder itself. It will be appreciated by those of skill in the art that these treatment purposes are often related and that treatments can be tailored for particular patients based on various factors. These factors can include the age, gender, or health of the patient, and the progression of the autoimmune or immune related disease or disorder. The treatment methodology for a patient can be tailored accordingly for dosage, timing of administration, route of administration, and by concurrent or sequential administration of other therapies.

The following are provided for illustrative purposes only, and are in no way intended to limit the scope of the present invention.

In one exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramulseular (i.m.) injection of about 20 million cells of the expanded CD8αα⁺, TCRαβ⁺, CD200⁺, CD122⁺ T_{reg} cell population in 1.0 ml phosphate buffered saline to treat Crohn's disease. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat transplant rejection. This dosage can be adjusted based on the results of the treatment and the judgment of the attending physician. Treatment is preferably continued for at least about 1 or 2 weeks, preferably at least about 1 or 2 months, and may be continued on a chronic basis.

In yet another exemplary embodiment, blood is drawn from a 70 kg adult patient. PBLs are isolated from the blood and CD8αα⁺, TCRαβ⁺, CD200⁺, CD122⁺T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat multiple sclerosis. This dosage can be adjusted based on the results of the treatment and the judgment of the attending physician. Treatment is preferably continued for at least about 1 or 2 weeks, preferably at least about 1 or 2 months, and may be continued on a chronic basis.

In still another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ Trig cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat systemic lupus erythematosus. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat Alzheimer's disease. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat rheumatoid arthritis. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat psoriatic arthritis. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat enterogenic spondyloarthropathies. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD88αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ T_{reg} cell population in 1.0 ml phosphate buffered saline to treat autoimmune hepatitis. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramuscular (i.m.) injection of about 20 million cells of the expanded CD8αα⁺, TCRαβ⁺, CD200⁺, CD122⁺ Tree cell population in 1.0 ml phosphate buffered saline to treat celiac disease. This dosage can be adjusted based on the results of the treatment and the judgment of the attending physician. Treatment is preferably continued for at least about 1 or 2 weeks, preferably at least about 1 or 2 months, and may be continued on a chronic basis.

In another exemplary embodiment, blood is drawn from a 70 kg adult patient. PBLs are isolated from the blood and CD8αα⁺, TCRαβ⁺, CD200⁺, CD122⁺ T_{reg} cells are then isolated from the PBLs. The CD8αα⁺, TCRαβ⁺, CD200⁺, CD122⁺ T_{reg} cell population is then expanded ex vivo on anti-CD3 coated plates in the presence of IL-2, IL-7 and IL-15. The patient is given a daily intramulseular (i.m.) injection of about 20 million cells of the expanded CD8αα⁺, TCRαβ⁺, CD200⁺, CD122⁺ T_{reg} cell population in 1.0 ml phosphate buffered saline to treat insulin dependent diabetes mellitus. This dosage can be adjusted based on the results of the treatment and the judgment of the attending physician. Treatment is preferably continued for at least about 1 or 2 weeks, preferably at least about 1 or 2 months, and may be continued on a chronic basis.

The following examples are provided for illustrative purposes only, and are in no way intended to limit the scope of the present invention.

### EXAMPLE 1

### Antigen Specificity of the CD8 T_{reg} Lines and Clones

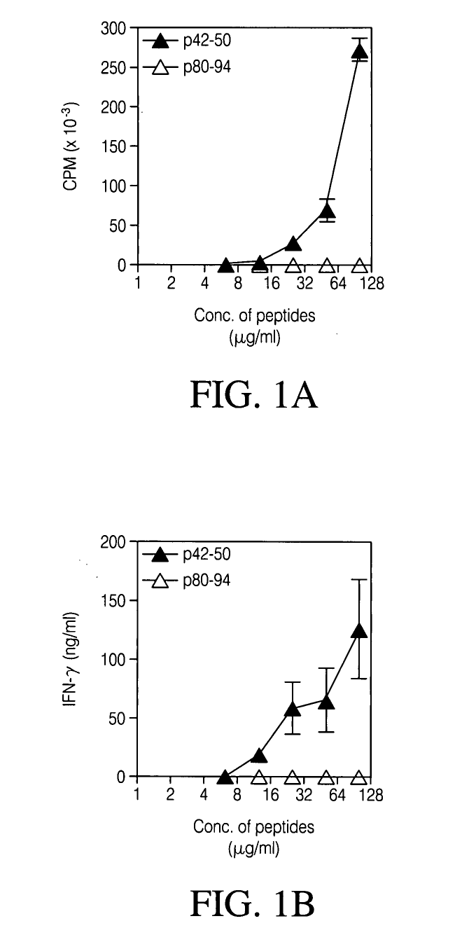

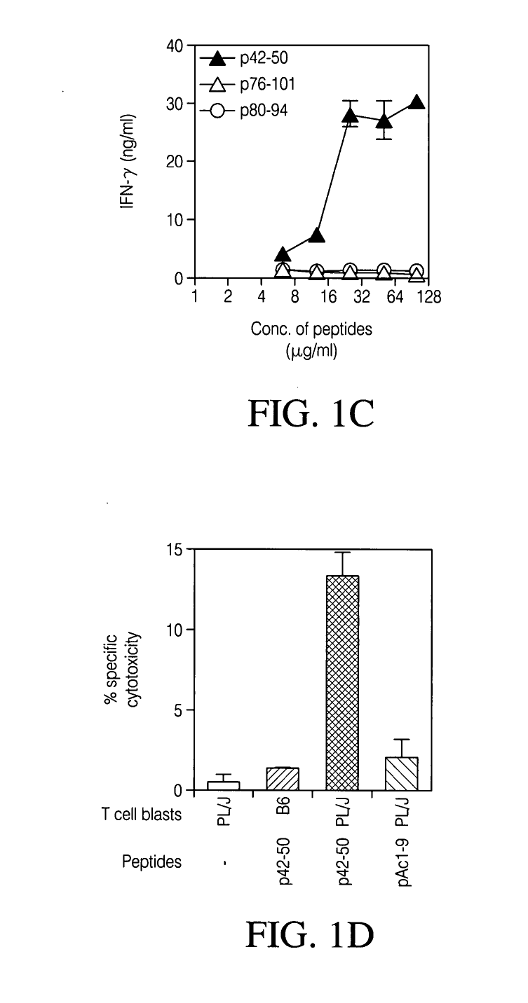

To examine antigen specificity, the CD8 T_{reg} lines and clones, peptide p42-50-reactive CD8 T cell lines and clones were generated by limiting dilution from lymph node cells of PL/J mice s.c immunized with p42-50 peptide (20 μg/mouse). Proliferative (FIG. 1a) and cytokine response (FIG. 1b) of representative CD8 T cell clone, 2D11 and a CD 8 T cell line (line #2) (FIG. 1c) are shown in FIG. 1. CD8 T cells (50,000) were incubated with p42-50 or control peptide p80-94 at titrated concentrations in the presence of 500,000 irradiated syngenic APCs. Thymidine incorporation was assayed following in vitro culture for 72 hr. Cytokine secretion was determined by the standard sandwich ELISA in supernatants from a 48 hr culture. (FIG. 1d) Specific cytotoxicity of the 2D11 CD8 T cell clone towards p42-50-pulsed targets was also recorded. ⁵¹Chromium-labeled blasts (10,000) from the syngenic PL/J or C57BL/6 mice pulsed with 10 μg/ml of p42-50 or irrelevant peptide Ac1-9 were incubated with the clone at an effector:target ratio of 30:1 for 4 hr. Supernatants were collected and chromium release was determined using a Trilux gamma counter. These data are representative of four independent experiments.

FIGS. 1a and 1b show that 2D11 proliferated and secreted IFN-γ in response to p42-50, but not to another peptide p80-94 derived from the TCR Vβ8.2 chain. Similarly a short-term CD8 T cell line responded to p42-50, but not to other Vβ8.2 chain-derived peptides (FIG. 1c). The cytotoxic capacity of the CD8 T_{reg} clones was determined using a standard 4 hr chromium release assay. Without being bound by a particular theory, FIG. 1d shows the killing of syngeneic ConA blasts pulsed with p42-50 but not an irrelevant peptide, the killing by the p42-50 CD8 T_{reg} clones and lines were MHC-restricted as it did not occur when allogeneic (BL/6) blasts were used as targets. See FIG. 1.

### EXAMPLE 2

### Phenotypic Analysis of a CD8 T_{reg} Clone

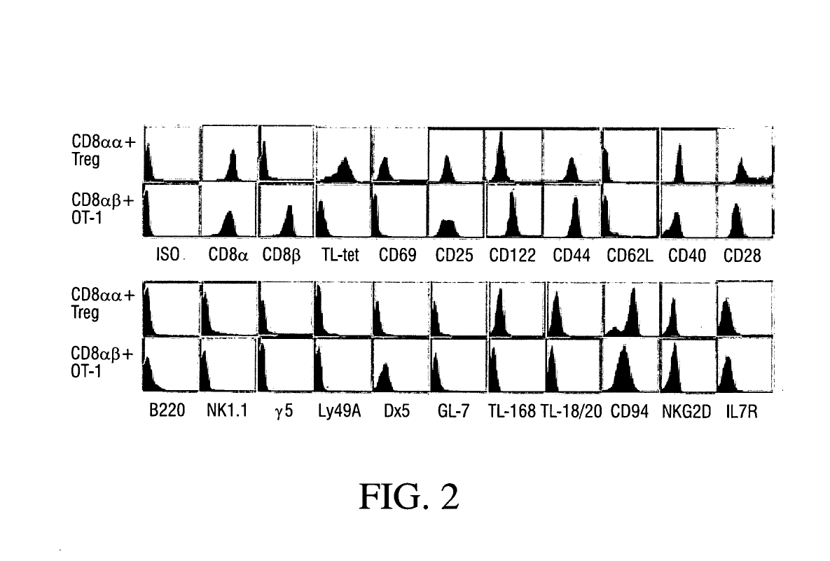

To determine whether CD8 T_{reg} express characteristic cell-surface markers, a panel of mAbs was used to stain 2D11. Staining was analyzed by flow cytometry. In parallel, a conventional CD8 T cell clone (OT-1) specific for a peptide of ovalbumin (SINFEKL) and propagated under equivalent conditions was used as a control. CD8 T_{reg} clone 2D11 and an irrelevant CD8 T cell clone were stained with the antibodies indicated in FIG. 2 (ISO, CD8a, CD8β, TL-tet, CD69, CD25, CD122, CD44, CD62L, CD40, CD28, B220, NK1.1, γδ, LY49A, Dx5, GL-7, TL-16B, TL-18/20, CD94, NKG2D and IL7R). Staining was analyzed by flow cytometry. These data are representative of three independent experiments.

As shown in FIG. 2, both clones express CD25 (IL-2Rac chain), CD122 (IL-2Rβ chain), and IL-7R, suggesting an activated/memory phenotype. The CD8 T_{reg} maintained low-level expression of CD69 even after a prolonged resting period in vitro in the absence of exogenous TCR peptide. Without wishing to be bound to a particular theory, these data indicate low-level of cross-presentation of p42-50 owing to the presence of Vβ8.2⁺ T cells in the irradiated splenocytes. The CD8 T_{reg} clones are negative for CD62L, NK1.1, Ly49A, γδ chain, GL-7, and B220 expression and positive for CD28, and thymic leukemia (TL) antigen expression.

It has been shown that CD8 T cells expressing a high level of CD94/NKG2 are relatively resistant to apoptosis compared to those with a null or intermediate level. Additionally, CD94/NKG2 receptors can interact with Qa-1/Qdm and provide survival signals for CD8 T cell maintenance in vivo. CD94 expression was examined (paired with NKG2A, B, C, and L to form a heterodimer) and NKG2D (homodimer) on 2D11 and the OT-1 clone. As shown in FIG. 2, although both clones were NKG2D⁺ and CD94⁺, the CD8 T_{reg} clone expressed higher levels of CD94, which may explain its relative resistance to apoptosis in vivo (see below).

One feature found in analyzing phenotype of the CD8 clones was the absence of surface CD8β chain expression (see FIG. 2). As thymic leukemia (TL) antigen displays a ten fold higher binding affinity for CD8αα homodimers compared to CD8αβ heterodimers, binding of 2D11 cells to TL tetramers was examined. As shown in FIG. 2, without being bound by a particular theory, only the CD8 T_{reg} clone bound to the TL-tetramer. It has been reported that CD8αα T cells do not easily adapt to long term in vitro culture. This may explain the difficulty in generating CD8αα⁺ T cell clones. See FIG. 2.

### EXAMPLE 3

### CD8 T_{reg} Clone 2D11 Secretes Tc-1-Like Cytokines and Kills Activated Vβ8.2⁺ CD4⁺ T Cells

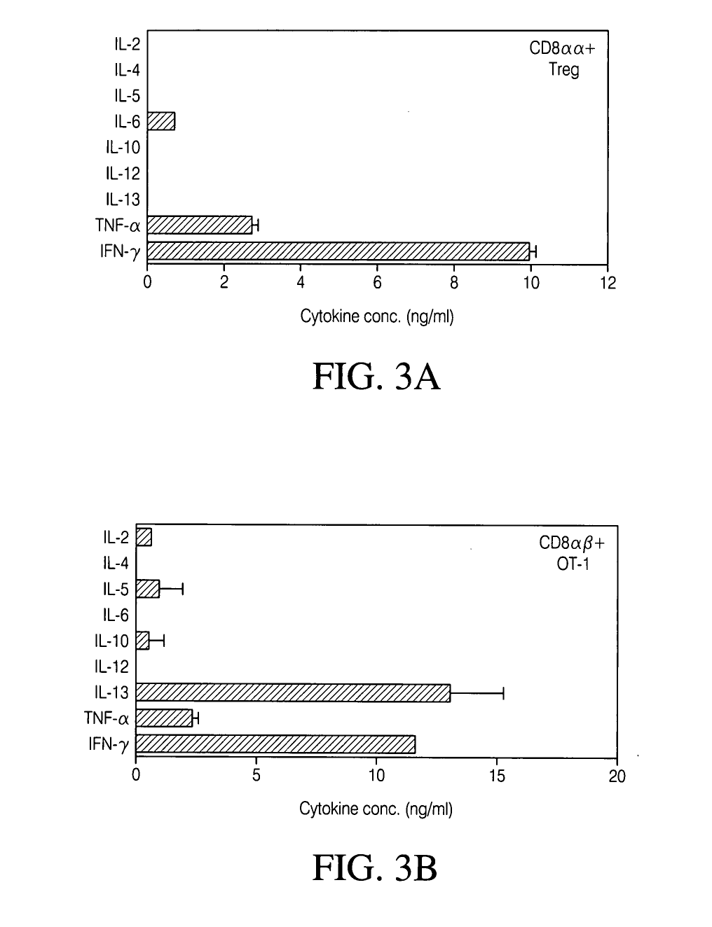

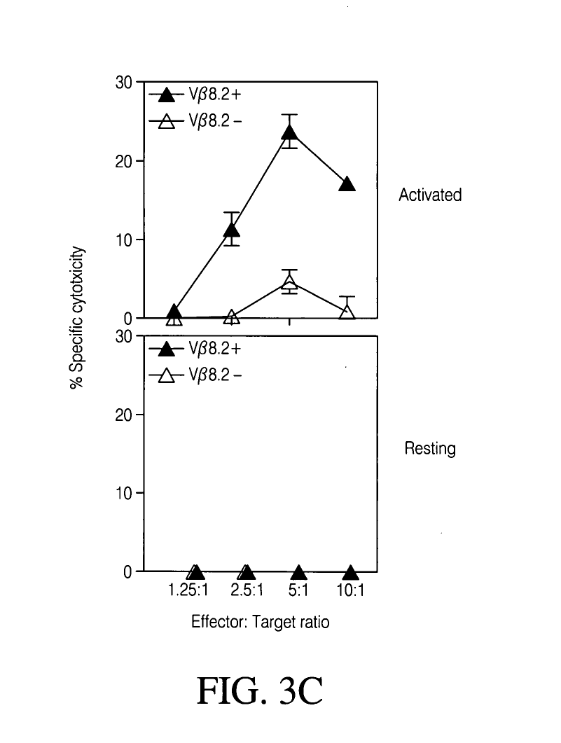

The cytokine secretion profile of 2D11 as well as an OT-1 ovalbumin-reactive CD8 clone (control) was examined in cell cultures after stimulation with peptide-pulsed APC. As shown in FIG. 3a, 2D11 secreted IFN-γ and TNF-α (Tc1-like), but no detectable level of IL-2, IL-4, IL-5, IL-10, IL-12, and IL-13. A very low level of IL-6 secretion was detected. In contrast, the OT-1 clone secreted IFN-γ, TNF-α, IL-2, IL-5, IL-10, and IL-13. The Tc1 phenotype of the T_{reg} clone was not an artifact of long-term culture because short-term p42-50-reactive T cell lines also secreted IFN-y, but not Tc2-like cytokines (Data not shown).

CD8 T cell-dependent depletion of activated, but not resting Vβ8.2⁺ CD4 T cells following induction of regulation has been shown in vivo. To determine whether CD8 T_{reg} clones could specifically kill Vβ8.2⁺ CD4 T cells, an MBPAc1-9-reactive pathogenic Vβ8.2⁺ T cell clone was used as a target in an in vitro cytotoxicity assay. In parallel, a Vβ14⁺ CD4 T cell clone was used as a Vβ8.2− target. As shown in FIG. 3c, 2D11 killed antigen-activated Vβ8.2⁺, but not Vβ14⁺ T cells (upper panel). To determine whether Vβ8.2⁺ T cell activation was required for killing, the CD4 T cell clones were rested until the cells had the small rounded appearance of naive T cells. No detectable cytotoxicity towards the resting CD4 T cell targets was found (lower panel in FIG. 3c).

Culture supernatants from 2D11 (FIG. 3a) and an OVA-reactive CD8 clone (FIG. 3b) were assayed for cytokines using Sandwich ELISA. Activated or resting Vβ8.2⁺ or Vβ14⁺ CD4 T cells were labeled with ⁵¹Chromium (10,000 target cells) and incubated with 2D11 at the indicated effector. target ratio (FIG. 3c). These data are representative of three independent experiments. See FIG. 3.

### EXAMPLE 4

### The CD8αα T_{reg} are Restricted by MHC Class Ib, Qa-1a Molecules

Genetic, biochemical, and immunological approaches were used to determine the MHC-restriction of the CD8 T_{reg} clones. First, in vitro proliferation assays were performed to examine the response of 2D11 to p42-50-pulsed APCs derived from a variety of mouse strains. As shown in Table 1, 2D11 responds to p42-50 pulsed APCs from PL/J (H-2^{u}), B10.PL (H-2^{u}), NZB (H-2^{d}), B10.BR (H-2^{k}), SWR/J (H-2^{q}), and NOD (H-2^{g7}) mice, but not to p42-50 pulsed APCs from C57BL/6 (H-2^{b}), BALB/c (H-2^{d}), and SJL/J (H-2^{s}) mice. Since MHC class Ia genes are different among these APCs, we reasoned that MHC class Ib molecules might be involved in the presentation of p42-50. Indeed, APCs capable of stimulating 2D11 expressed the Qa-1a allele, whereas those incapable of presentation expressed the Qa-1b allele. Without wishing to be limited to any particular theory, these data suggest that Qa-1a molecules present p42-50 to the CD8 T_{reg}. This is consistent with the finding that B10.PL and PL/J mice from which the CD8 T_{reg} clones were isolated, express Qa-1a but not Qa-1b molecules (Data not shown).

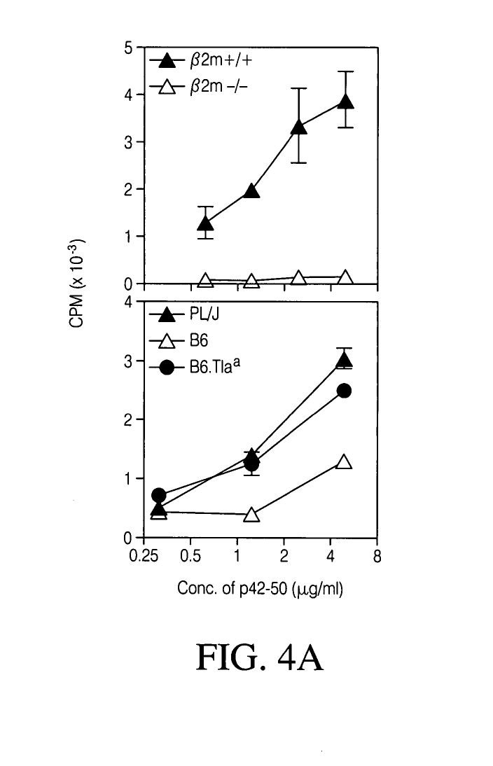

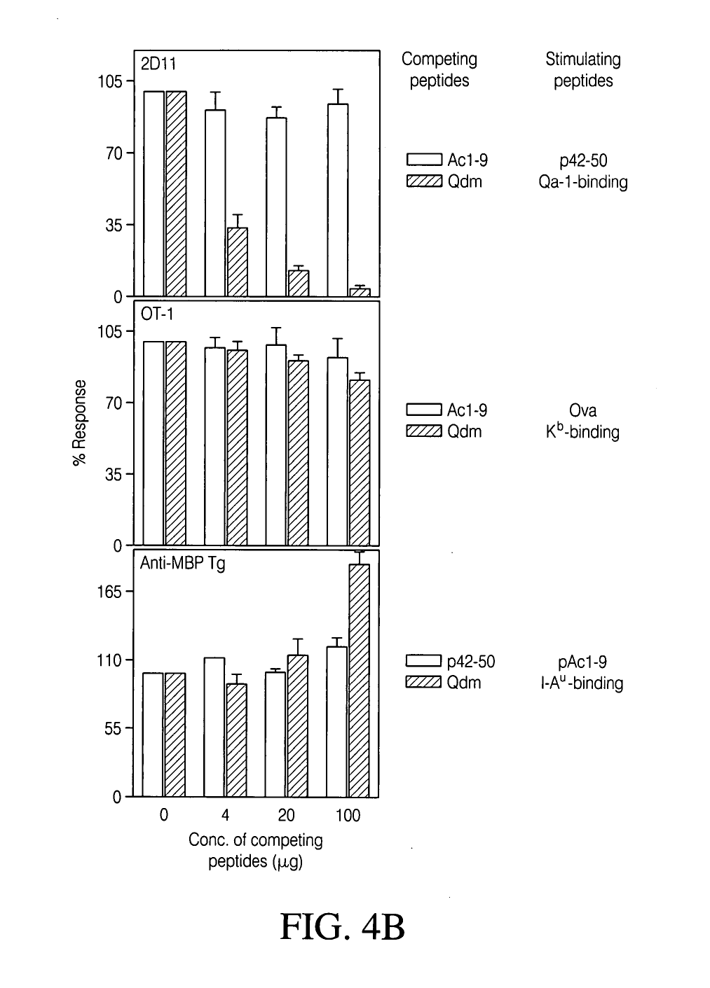

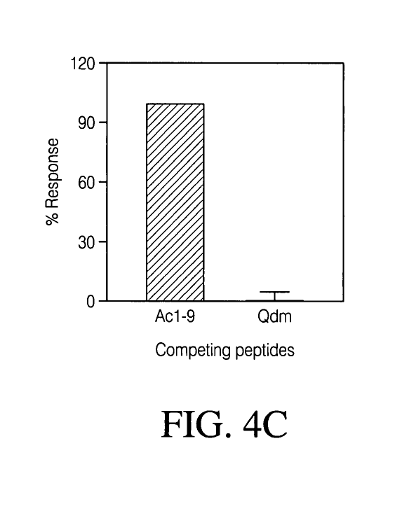

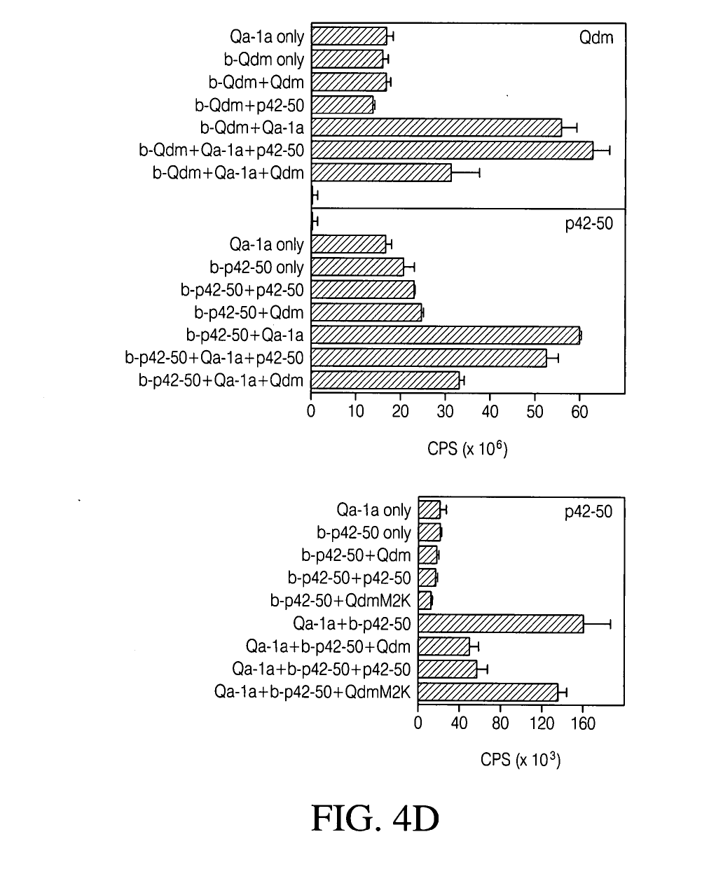

To determine whether presentation of p42-50 requires β2m which pairs with a heavy chain to form a MHC class I molecule, the ability of APCs from both β2 m^{+/+} and β2m^{-/-} mice to stimulate 2D11 in vitro was compared. As shown in the upper panel of FIG. 4a, β2m^{+/+} but not β2m^{-/-} APCs were able to present p42-50 and stimulated the 2D11 clone. To further validate Qa-1a presentation, a comparison was done between the presentation of p42-50 by Qa-1 congenic mice B6.Tla^{a} (Qa-1a) and B6 (Qa-1b). Data in the lower panel of FIG. 4a show that APCs from B6.Tla^{a}, but not B6, can present peptide p42-50. Likewise, the 2D11 clone showed specific cytotoxicity towards p42-50-pulsed T2 Qa-1a transfectants, but not T2 cells (Data not shown).

The canonic Qa-1-binding peptide Qdm (Qa-1 determinant modifier) has been shown to bind with high affinity to both Qa-1a and Qa-1b molecules. It was tested whether Qdm could compete with p42-50 in stimulation of the 2D11 clone. The upper panel of FIG. 4b shows a dose-dependent inhibition of 2D11 proliferation in the presence of Qdm peptide (black bar), but not in the presence of an irrelevant class II-binding peptide MBPAc1-9 (white bar) or class Ia-binding ovalbumin peptide (data not shown). In contrast, responses of the OT-1 clone (class Ia-restricted, middle panel) and an MBPAc1-9-reactive CD4 T cell clone (class II-restricted, lower panel) were not blocked by the Qdm peptide. To exclude the possibility that Qa-1 restriction was not just a property of this particular 2D11 clone, it was tested whether the Qdm peptide could block a bulk recall response to p42-50. As shown in FIG. 4c, Qdm, but not an irrelevant peptide blocks the p42-50 response of draining lymph node cells derived from p42-50 immunized mice.

The above experiments indicate that p42-50 is presented by the Qa-1a molecule to the CD8 T_{reg}. To directly demonstrate binding of p42-50 to Qa-1a molecules, a binding assay was performed using purified recombinant Qa-1a molecules. Both peptides p42-50 and Qdm were biotinylated (b-p42-50 and b-Qdm), and a fluorescence tag was added for their visualization and quantification in an in vitro binding assay as described earlier¹⁹. As shown in the upper panel of FIG. 4d, both b-Qdm and b-p42-50 bind to Qa-1a, and its binding is competed by unlabeled Qdm (100 μM). A ten fold higher concentration (10 μM) of b-p42-50, compared to b-Qdm was required for comparable binding to Qa-1a. This suggests that p42-50 may have either a lower binding affinity or a higher off-rate compared to Qdm. Accordingly a two fold higher concentration (200 μM) of unlabeled p42-50 was necessary for blocking the binding of b-p42-50 to Qa-1a molecules (lower panel of the FIG. 4d).

The Proliferative response of the CD8 T_{reg} clone 2D11 in the presence of p42-50-pulsed irradiated APCs from β2m^{+/+} and β2m^{-/-} mice is shown in FIG. 4a, upper panel. Data are representative of two independent experiments. In the lower panel, the proliferative response of 2D11 to p42-50 at titrated doses in the presence of APCs derived from syngenic PL/J, allogeneic C57BL/6, or congenic B6.Tla^{a} mice is shown. Data are representative of three independent experiments. 2D11 cells were cultured at an optimal concentration of p42-50 (0.625 μg/ml) in the presence of increasing concentrations of blocking peptide Qdm or a control peptide Ac1-9 (See FIG. 4b, upper panel). The proliferative response of the 2D11 was blocked by Qdm but not Ac1-9. Shown in FIG. 4b, middle and lower panels are proliferative responses of OVA-reactive CD8 T cell clone (middle panel) and Ac1-9-reactive CD4 T cell clone (lower panel) to their respective peptides were not blocked by the presence of Qdm peptide. Data are representative of three independent experiments. FIG. 4c shows that the proliferative response to p42-50 in draining lymph node cells isolated from p42-50-immunized mice was inhibited in the presence of Qdm but not in the presence of AC1-9. Data are representative of two independent experiments. FIG. 4d shows the binding of biotinylated p42-50 peptide to purified Qa1-a molecules. Purified Qa-1a/Qdm complexes were incubated overnight with 1 μM biotin-Qdm4C (b-Qdm) or 10 μM biotin-p42-50-4C (b-p42-50) in the presence or absence of 100 μM (upper panel) or 200 μM (lower panel) unlabeled competing peptides (p42-50, Qdm, or QdmM2K). Complexes were then separated from unbound peptides and the amount of biotinylated peptides bound to Qa-1a was measured by europium-based fluorescence immunoassay using an anti-β2m capture antibody. QdmM2K is a negative control peptide with a substitution of Methionine with Lysine at β2 leading to the loss of binding to Qa-1a. Data are representative of at least three independent experiments.

### EXAMPLE 5

### Adoptive Transfer of CD8 T_{reg} Lines and Clones Results in Quick Recovery and Protection from EAE

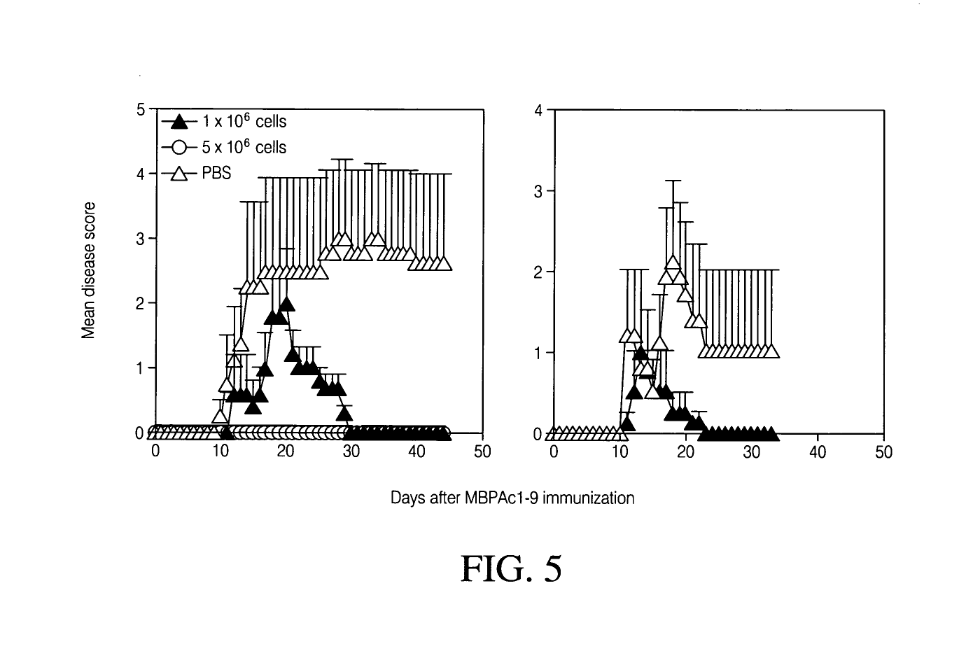

To determine the in vivo regulatory potential of the CD8 T_{reg}, 2D11 cells were adoptively transferred intravenously into syngeneic mice. After 24 hours, recipients were immunized with MBPAc1-9/CFA/PT for the induction of EAE, and clinical symptoms were monitored daily and scored for 35-45 days on a scale from 1-5:1 being tail paralysis; 2 being hind limb paralysis; 3 being hind body paralysis; 4 being hind limb and partial body paralysis; and 5 being whole body paralysis or moribund. As shown in the right panel of FIG. 5, mice injected with 1 million 2D11 cells recover more rapidly from a milder disease than those in the control group. To rule out the possibility that the ability to control disease is an artifact of the long-term culture of a particular clone, short-term p42-50-reactive T cell lines were generated and used in similar adoptive transfer experiments. As shown in the left panel of the FIG. 5, mice that receive five million cells of a p42-50-reactive T cell line are completely protected from MBP-induced EAE, and transfer of only one million cells enables a more rapid recovery from a milder paralysis. Adoptive transfer of short-term T cell lines raised against two irrelevant TCR peptides had no effect on EAE (data not shown). See FIG. 5.

### EXAMPLE 6

### p42-50-Reactive CD8 T_{reg} Predominantly Use the TCR Vβ6 Gene Segment

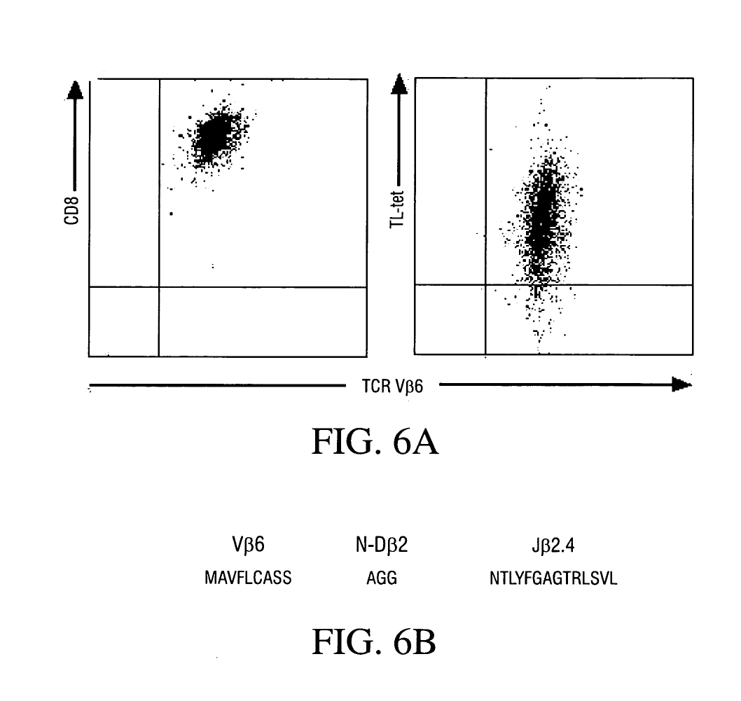

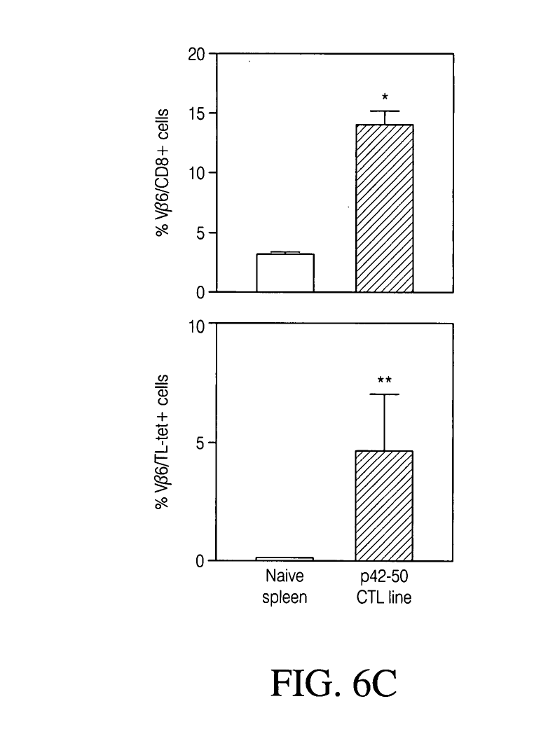

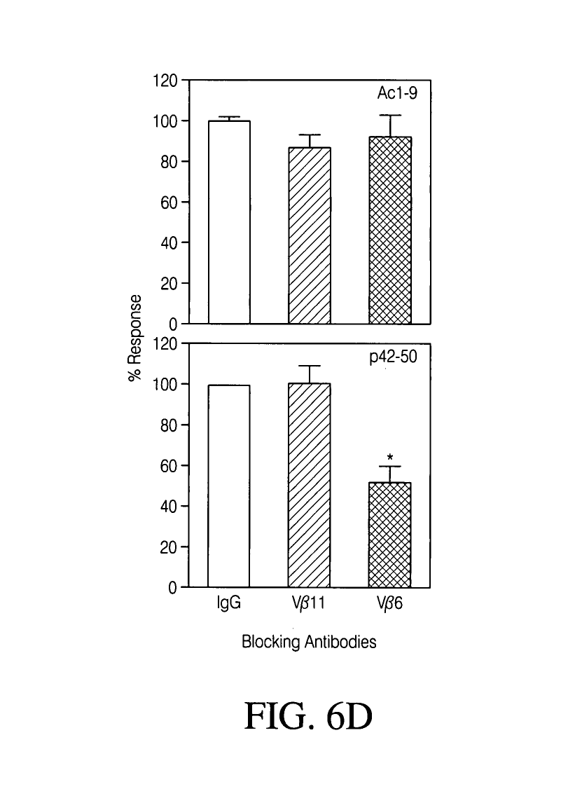

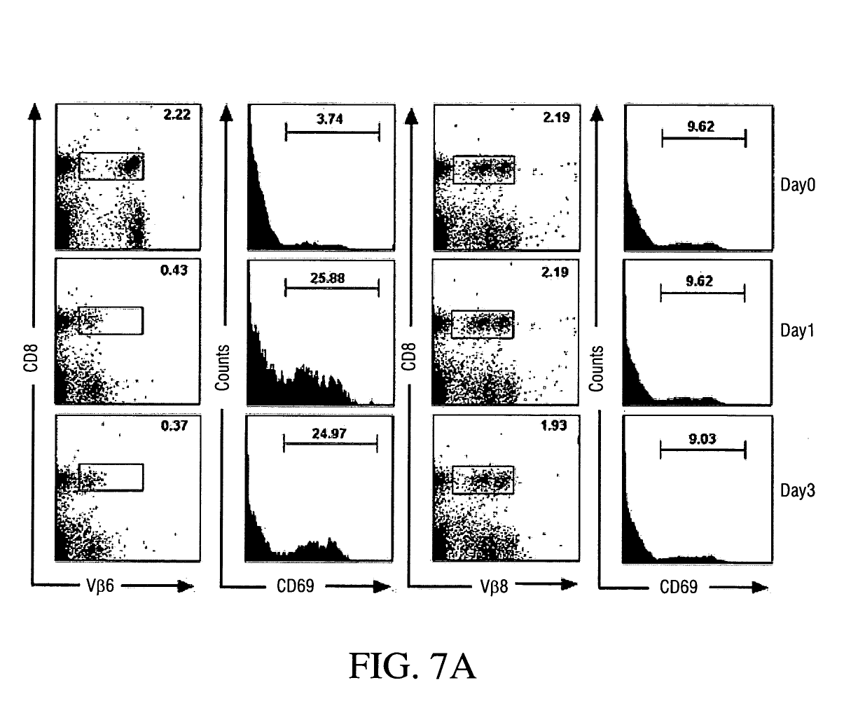

To determine the TCR Vβ gene usage of the CD8 T_{reg} clones, cells were stained with a panel of anti-TCR Vβ mAbs and analyzed by flow cytometry. Two out of the three CD8 clones, including 2D11 utilized Vβ6 (left panel of FIG. 6a) and displayed CD8αα+ homodimers on the cell surface (right panel). Sequencing the CDR3 region of 2D11 revealed usage of Dβ2 and Jβ2.4 gene segments (FIG. 6b). Using DNA spectratyping analysis, we confirmed that two of the three CD8 T_{reg} clones, which were isolated from different animals, used a similar CDR3 region in the TCR Vβ chain. This phenomenon was also observed in the CD4 T_{reg} population in this system.

It was hypothesized that the T_{reg} population was oligoclonal with respect to TCR usage. To determine whether the TCR Vβ6 gene segment is predominantly utilized by p42-50-reactive CD8 T cells, short-term CD8 T cell lines were generated and stained with anti-TCR Vβ6-FITC and anti-CD8α-PE or TL tetramer-PE, and analyzed by flow cytometry. As shown in FIG. 6c, Vβ6⁺ CD8αα⁺ T cells were significantly expanded in the lines (Vβ6⁺ CD8α⁺ in the upper panel, and Vβ6⁺TL-teramer⁺ cells in the lower panel). It was then determined whether an anti-TCR Vβ6 blocking antibody was able to inhibit an ex vivo recall response to p42-50. Without being bound by a particular theory, as shown in the lower panel of FIG. 6d, the p42-50 recall response was significantly suppressed in the presence of anti-TCR Vβ6 mAb, but not in the presence of an isotype control or an irrelevant mAb (anti-TCR Vβ11). In confirmation of the specificity of the inhibition, a recall response to a peptide not predominantly using TCR Vβ6⁺ T cells was not blocked by the anti-Vβ6 mAb (FIG. 6d, upper panel).

As shown in FIG. 6a, 2D11 is CD8αα⁺ and Vβ6⁺ as demonstrated by staining with TCR, Vβ6-FITC and CD8α-PE, or TL-tetramer-PE. Data are representative of at least three independent experiments. In FIG. 6b, the CDR3 region gene sequence of the Vβ chain expressed by the CD8 T_{reg} clone, 2D11 is shown. FIG. 6c shows the expansion of Vβ6⁺, TL-tetramer⁺, and CD8⁺ T cells in short-term cell lines reactive to p42-50. Cells were stained with TCR Vβ6-FITC/CD8α-PE (upper panel) or TCR Vβ6-FITC/TL-tetramer-PE (lower panel). P values were *P<0.0001, **P<0.05. As shown in FIG. 6d, the immune response was present to p42-50 (lower panel) but not to the irrelevant peptide, Ac1-9 (upper panel) was significantly inhibited in vitro in the presence of anti-Vβ6 mAb but not in the presence of an isotype control or irrelevant anti-TCR Vβ11 mAb. Results are shown as a percentage of the response recorded when the IgG isotype control was used. *P<0.05 as compared with IgG or anti-TCR Vβ11 group; ANOVA test. See FIG. 6.

### EXAMPLE 7

### Activation of TCR Vβ6⁺ T Cells In Vivo Leads to Protection from EAE

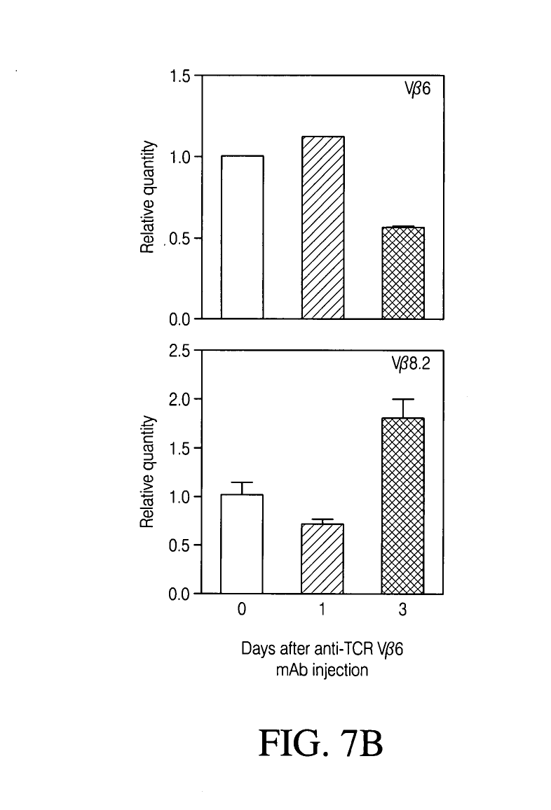

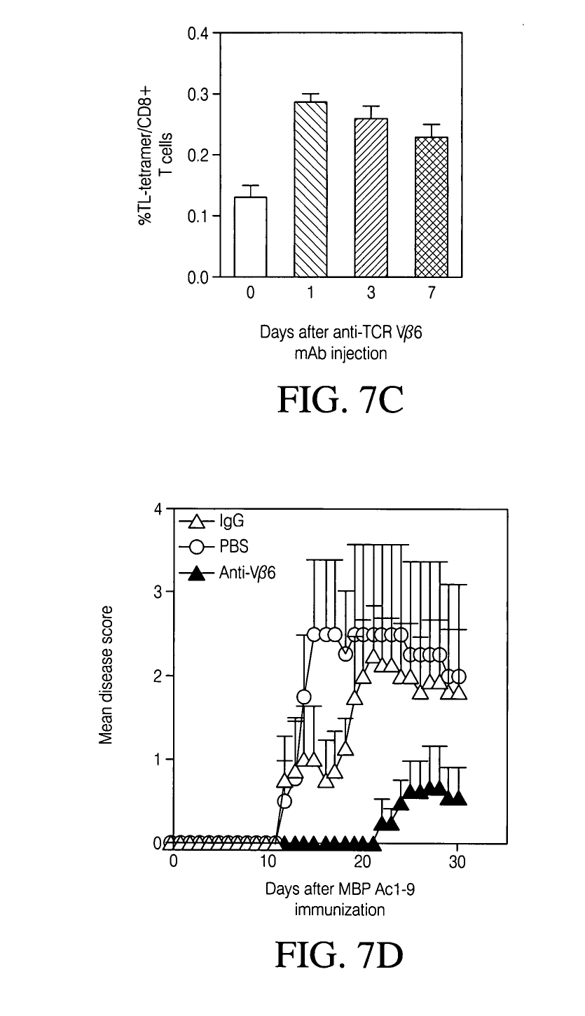

The predominant usage of the TCR Vβ6 chain by the CD8 T_{reg} is shown by an examination of whether treatment of mice with anti-Vβ6 mAb had any influence on the CD8 T_{reg} repertoire and the course of MBPAc1-9-induced EAE. Following a single intravenous injection of anti-TCR Vβ6 mAb (RR4-7, 300 μg/mouse), splenocytes were examined at different times for the presence of Vβ6⁺ cells in vivo. As expected, anti-TCR Vβ6 mAb injection led to early activation of TCR Vβ6⁺ T cells in vivo as determined by the expression of the early activation marker CD69 (FIG. 7a, second column), followed by down-regulation of cell-surface Vβ6 expression (FIG. 7a, first column). The effect of the anti-TCR Vβ6 mAb was TCR-specific because Vβ8⁺ T cells were not affected under identical experimental conditions (FIG. 7a, third and fourth columns). Real-time RT-PCR analysis of TCR Vβ6 mRNA expression in splenocytes from anti-TCR Vβ6-treated mice revealed that most but not all of the TCR Vβ6⁺ T cells were depleted following continuous treatment (FIG. 7b and data not shown).

CD8αα T cells predominantly present in intraepithelial lymphocytes (IEL) in the intestine are relatively resistant to deletion by self-agonists and might differ from CD8αβ T cells in their response to activation through the T cell receptor. It was examined whether anti-TCR Vβ6 mAb administration led to deletion or activation of the CD8αα T cells in the periphery using staining with the TL-tetramer. The data in FIG. 7c show that TL-tetramer⁺ CD8⁺ cells are not depleted following antibody administration. In fact, a slight increase in an in vitro recall response to p42-50 was found in antibody-treated mice (data not shown). To further test the regulatory function after activation of the CD8αα⁺ Vβ6⁺ T cells, groups of mice were immunized with MBPAc1-9/CFA/PT for the induction of EAE. One day and one week later a low dose of either the anti-TCR Vβ6 mAb (100 μg/mouse), an isotype control mAb (100 μg/mouse), or PBS was injected intravenously. Clinical symptoms were monitored daily after the second injection. The data in FIG. 7d and in Table 2 show that in vivo activation of Vβ6⁺ T cells with the anti-TCR Vβ6 mAb resulted in protection from EAE in B10.PL mice. In contrast, animals in the group injected with the isotype control mAb or an irrelevant mAb were not protected from disease (data not shown).

Activation (CD69 expression) followed by down-regulation/depletion of Vβ6⁺, but not Vβ8⁺ T cells after a single intravenous injection with anti-TCR Vβ6 mAbs (300 μg/mouse). At days 0, 1, and 3, splenocytes were stained with TCR Vβ6-FITC/CD8α-PE/CD69-PerCP (first and second columns) or TCR Vβ8-FITC/CD8α-PE/CD69-PerCP (third and fourth columns). The cells were gated on either TCR Vβ6/CD8α⁺ T cells (second column) or TCR Vβ8/CD8α⁺ T cells (fourth column) to analyze the expression of the early activation marker CD69. Data are representative of two independent experiments. (b) Significant depletion of Vβ6⁺ T cells following anti-Vβ6 injection as determined by Real-Time PCR. Expression of mRNA is presented as relative quantity after normalization against two internal control genes (L32 and Cyclophilin). Data are representative of two independent experiments. (c) TL-tetramer⁺ CD8 T cells are not deleted following anti-TCR Vβ6 injection. Splenocytes were stained with CD88α-FITC/TL-tetramer-PE on the indicated days following antibody injection. Data are representative of two independent experiments. (d) Mice injected with anti-TCR Vβ6 mAb are protected from EAE. One and seven days after the induction of EAE with MBPAc1-9/CFA/PT, groups of mice were injected with anti-TCR Vβ6 mAb (100 μg/mouse) or an IgG isotype control or PBS. Paralytic disease was monitored as described in the Methods. Data are combined from three independent experiments.

TABLE 1
Response profile of the CD8αα+ T cell clone¹

| | | H-2K | H-2D | H-2L | Qa-1 | Qa-2 | Response |
|---|---|---|---|---|---|---|---|
| PL/J^{(u)2} | | u | d | d | a | ? | + |
| B10.PL-H2u | H2- | u | d | d | a | ? | + |
| T18a(73NS)SnJ^{(u)} | | | | | | | |
| NZB/BINJ^{(d)} | | d | d | — | a | a(+) | + |
| B10.BR-H2k | H2- | k | k | k | a | | + |
| T18a/SgSnJ^{(k)} | | | | | | | |
| SWR/J^{(q)} | | q | q | q | a | a(+) | + |
| NOD.ALR- | | d | b | | a | ? | + |
| (D17Mit16- | | | | | | | |
| D17Mit10)NOD/ | | | | | | | |
| Lt^{(g7)} | | | | | | | |
| C57BL/6J^{(b)} | | b | b | — | b | a(hi) | − |
| BALB/cJ^{(d)} | | d | d | d | b | a(lo) | − |
| SJL/J^{(s)} | | s | s | | b | a | − |

¹The CD8αα T_{reg} clone 2D11 was examined for proliferation or cytokine secretion in response to p42-50 in the presence of antigen presenting cells (APCs) derived from different H-2 haplotyes. The H-2K, D, L, Qa-1, and Qa-2 haplotypes are shown.
²H-2 haplotypes.
3. “+” and “−” in the last column refer to a positive response and no response to p42-50, respectively. Positive response is defined as stimulating indices (SI) greater than 3 (7.25 +/− 1.93 to 58.06 +/− 18.69).

TABLE 2
Activation of TCR Vβ6⁺ T cells leads to protection from EAE

| | Incidence of EAE | Mean days |
|---|---|---|
| | # of animals with disease/total # of animals | of |
| Antibodies¹ | (maximal individual disease scores) | disease onset |
| PBS | 3/4 (5, 3, 3, 0) | 13.3 +/− 1.2 |
| IgG | 10/12 (5, 5, 4, 3, 3, 3, 3, 2, 1, 1, 0, 0) | 14.8 +/− 3.2 |
| Anti-Vβ3 | 6/6 (5, 5, 4, 4, 2, 1) | 13.1 +/− 2.2 |
| Anti-Vβ6 | 5/12 (5, 4, 2, 1, 1, 0, 0, 0, 0, 0, 0, 0) | 26.7 +/− 5.5 |

¹Antibodies (100 μg) were injected I.V. on Day 1 and Day 8 following MBPAc1-9 immunization (Day 0).

### EQUIVALENTS

The foregoing written specification is considered to be sufficient to enable one skilled in the art to practice the invention. The foregoing description details certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method of treating, preventing, or delaying the onset of an autoimmune disease in a patient comprising:
administering isolated CD8αα⁺, TCRαβ⁺ T cells to the patient.

2. The method of claim 1, further comprising:
obtaining a cell sample from a mammal;
isolating CD8αα⁺, TCRαβ⁺ T cells from the cell sample; and
expanding the isolated T cells.

3. The method of claim 1, wherein the isolated T cells are CD8αα⁺, TCRαβ⁺, CD200⁺.

4. The method of claim 1, wherein the isolated T cells are CD8αα⁺, TCRαβ⁺, CD122⁺.

5. The method of claim 1, wherein the isolated T cells are CD8αα⁺, TCRαβ⁺, CD200⁺, CD122⁺.

6. The method of claim 1, wherein the isolated T cells are a mixture of two or more of CD8αα⁺, TCRαβ⁺ T cells, CD8αα⁺, TCRαβ⁺, CD200⁺ T cells, CD8αα⁺, TCRαβ⁺, CD122⁺ T cells and CD8αα⁺, TCRαβ⁺, CD200⁺ CD122⁺ T cells.

7. The method of claim 2, wherein the cell sample comprises a blood sample.

8. The method of claim 2, wherein the cell sample comprises a tissue sample.

9. The method of claim 2, wherein the cell sample comprises lymph tissue.

10. The method of claim 2, wherein the mammal is the patient.

11. The method of claim 2, wherein the mammal is not the patient.

12. The method of claim 1, wherein the autoimmune disease is selected from the group consisting of multiple sclerosis, Crohn's disease, systemic lupus erythematosus, Alzheimer's disease, rheumatoid arthritis, psoriatic arthritis, enterogenic spondyloarthropathies, insulin dependent diabetes mellitus, autoimmune hepatitis, transplant rejection and celiac disease.

13. The method of claim 1, wherein the isolated T cells are isolated using antibodies.

14. The method of claim 13, wherein the antibodies are at least one of anti-TCR, anti-CD8αα, anti-CD200 and anti-CD122.

15. The method of claim 1, wherein the isolated T cells are expanded with growth factors.

16. The method of claim 1, wherein the isolated T cells are expanded with agents comprising anti-CD3 coated plates and one or more of IL-2, IL-7 and IL-15.

17. The method of claim 1, wherein the autoimmune disease is multiple sclerosis.

18. The method of claim 1, wherein the autoimmune disease is transplant rejection.

19. The method of claim 1, wherein the T cells are administered to the patient by one or more of the routes consisting of intravenous, intraperitoneal, intramuscular, subcutaneous, nasal and oral.

20. The method of claim 1, wherein the T cells are administered to the patient by an intramuscular route.

21. The method of claim 1, wherein the patient is human.

22. The method of claim 1, wherein the T cells administered to the patient comprise about 20 million cells.

23. An isolated T cell population, comprising an isolated population of T_{reg} cells characterized as CD8αα⁺, TCRαβ⁺.

24. The isolated T cell population of claim 23, wherein the T cell population is CD200⁺.

25. The isolated T cell population of claim 23, wherein the T cell population is CD122⁺.

26. The isolated T cell population of claim 23, wherein the T cell population is CD200⁺, CD122⁺.

27. The isolated T cell population of claim 23, further in combination with an aqueous vehicle and an additional pharmaceutically acceptable excipient.

28. The isolated T cell population of claim 24, further in combination with an aqueous vehicle and an additional pharmaceutically acceptable excipient.

29. The isolated T cell population of claim 25, further in combination with an aqueous vehicle and an additional pharmaceutically acceptable excipient.

30. The isolated T cell population of claim 26, further in combination with an aqueous vehicle and an additional pharmaceutically acceptable excipient.
